(19) United States
(12) Patent Application Publication
(10) Pub. No.: US 2004/0090797 A1
(43) Pub. Date: May 13, 2004

(54) Illuminated maraca

(76) Inventor: Wayne Cohen, Bangkok (TH)

Correspondence Address: BAKER & BOTTS, 30 ROCKEFELLER PLAZA, NEW YORK, NY, 10112

(21) Appl. No.: 10/399,074

(22) PCT Filed: Jan. 30, 2003
(86) PCT No.: PCT/US03/02816

(51) Int. Cl. A61B 1/24

(52) U.S. Cl. 362/577

(57) ABSTRACT

An illuminated maraca has a transparent shell and a handle. A light is arranged within the shell and operated by a circuit in the handle. In a preferred arrangement the circuit is operative in response to movement of the maraca to operate the light.

### CROSS REFERENCE TO RELATED APPLICATIONS

This application claims the benefit of U.S. Provisional Application Serial No. 60/355,505 filed Feb. 7, 2002.

### BACKGROUND OF THE INVENTION

This invention relates to novelty items and particularly to maracas, which may be used as amusement and promotional items for resorts, cruise ships, night clubs and parties, as well as for toys.

It is an object of the invention to provide a new and improved maraca novelty item that is attractive and conducive to party spirit.

### SUMMARY OF THE INVENTION

In accordance with the invention there is provided an illuminated maraca, having a translucent shell, a handle connected to the shell and a light within the shell. A circuit, including a battery, is arranged in the handle for operating the light. Pellets are provided within the shell for sound making.

The maraca preferrably includes a translucent shield surrounding the light. In a preferred embodiment the circuit includes a motion sensor for operating the light in response to motion of the illuminated maraca, such as shaking. The circuit may also include an oscillator circuit for causing the light to flash. A timing circuit is optionally provided for activating the oscillator circuit and light for a selected period of time in response to activation of the motion detector. A switch may be provided, and in one arrangement the handle includes a rotatable end cap which activates the switch.

For a better understanding of the present invention together with other and further objects thereof, reference is made to the following description, taken in conjunction with the accompanying drawing, and its scope will be pointed out in the appended claims.

### BRIEF DESCRIPTION OF THE DRAWINGS

FIG. 1 is an elevation plan view of an exemplary embodiment of the invention.

FIG. 2 is a first central cross sectional view thereof.

FIG. 3 is a second perpendicular cross sectional view thereof.

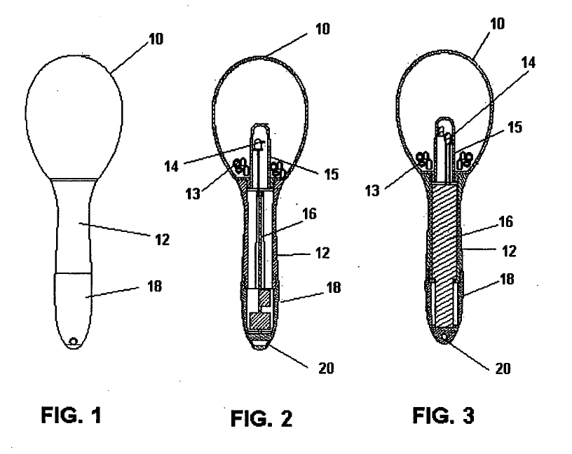

### DESCRIPTION OF THE INVENTION

The drawings illustrate plan elevation and cross-sectional views of an embodiment of the invention. The embodiment is a maraca which includes a hollow shell 10 and a handle 12. As seen in the cross-sectional views of FIGS. 2 and 3, sounding pellets 13 are provided within shell 10 to make a rattle sound when the maraca is shaken. Shell 10 is fabricated of translucent or transparent plastic material, preferrably of a hard material for improved sound.

A light 14 is provided within shell 10. In the embodiment illustrated, light 14 is an LED, which is preferred because of its small size, light weight and low power consumption. It is possible however to use a small light bulb to provide stronger illumination. A circuit board 16 is provided within handle 12 for operating light 14. In the embodiment shown handle 12 includes an end cap 18, which can be removeable to provide access to a battery or switch on circuit board 16. Alternately cap 18 may be permanently attached after assembly. Alternately handle 12 may be fabricated in one piece, including the end cap.

In a preferred arrangement light 14 is enclosed in inner shield 15 to protect it from the action of pellets 13 when the maraca is shaken. Shield 15 is translucent and preferrably transparent. Light 14 may be attached to the shield, for example using adhesive, to provide impact protection and mechanical strength.

In the embodiment illustrated a bore 20 is provided at the end of the handle cap 18 for attachment to a key chain or cord, for example to wear on the neck.

Light 14 may be activated by an internal or external switch. In one arrangement handle cap 18 is rotatable to activate a switch. Light 14 is powered by a small battery connected to circuit board 16. In one arrangement light 14 is activated by shaking of the maracca using a motion sensor on circuit boart 16. The circuit may provide a single short flash when shaken, or may include a timing circuit to keep the light illuminated or flashing for a selected period, such as two to twenty seconds after activation. Alternately the circuit may cause the light to flash on and off for a selected period after activation, such as five to sixty seconds with a flashing period of about one to two seconds. Circuits for providing such operation are known, for example in children's sneakers, and are available from Bliss Electronics of Taiwan. In another arrangement, circuit board 16 may include a microphone and a low pass filter for providing activation pulses for flashing the light in response to the low frequency beat of music such as disco music.

It should be understood that the shell 10 may be further decorated with a logo or design on the translucent or transparent plastic shell. The pattern on the exterior of the shell may be for advertising, may represent a sports team, a country, a resort or a state.

It should be further understood that the maraca can be made in a variety of sizes, as a small key chain or backpack ornament, for example 1.5 to 5 inches in overall length, as a full size maraca of about 6 to 9 inches in length or as an oversize maraca of larger length for use at a show or sporting event.

The lighted maraca of the present invention can provide combined sound and light to add to the excitement at a show, sporting event or on the dancefloor, particularly in conditions of low illumination.

While there have been described what are believed to be the preferred embodiments of the present invention, those skilled in the art will recognize that other and further changes and modifications may be made thereto without departing from the spirit of the invention and it is intended to claim all such changes and modifications as fall within the true scope of the invention.

I claim:

1. An illuminated maraca, comprising:
a translucent shell;
a handle connected to said shell;
a light within said shell;
a circuit, including a battery, arranged in said handle for operating said light; and
pellets in said shell.

2. An illuminated maraca as specified in claim 1 further including a translucent shield surrounding said light.

3. An illuminated maraca as specified in claim 2 wherein said light is attached to said shield.

4. An illuminated maraca as specified in claim 1 wherein said circuit includes a motion sensor for operating said light in response to motion of said illuminated maraca.

5. An illuminated maraca as specified in claim 4 wherein said circuit includes an oscillator circuit for causing said light to flash.

6. An illuminated maraca as specified in claim 5 wherein said circuit includes a timing circuit for activating said oscillator circuit and said light for a selected period of time in response to activation of said motion detector.

7. An illuminated maraca as specified in claim 1 wherein said circuit includes an oscillator circuit for causing said light to flash.

8. An illuminated maraca as specified in claim 7 wherein said circuit includes a switch.

9. An illuminated maraca as specified in claim 8 wherein said handle includes an end cap arranged for rotation with respect to the remainder of said handle, and wherein said rotation of said end cap activates said switch.
